(19) United States
(12) Patent Application Publication
(10) Pub. No.: US 2015/0099926 A1
(43) Pub. Date: Apr. 9, 2015

(54) Endoscope with Integrated Sensors

(71) Applicant: EndoChoice, Inc., Alpharetta, GA (US)

(72) Inventors: Tal Davidson, Yokneam Ilit (IL); Moshiko Levi, Ganey Tikva (IL); Idan Levy, Hadera (IL); Mark Gilreath, Alpharetta, GA (US)

(21) Appl. No.: 14/505,389

(22) Filed: Oct. 2, 2014

Related U.S. Application Data
(60) Provisional application No. 61/886,572, filed on Oct. 3, 2013.
(60) Provisional application No. 61/890,881, filed on Oct. 15, 2013.
(60) Provisional application No. 61/980,682, filed on Apr. 17, 2014.

(51) Int. Cl. A61B 1/00; A61B 1/31; A61B 1/04

(52) U.S. Cl. CPC A61B 1/00055; A61B 1/04; A61B 1/31; A61B 1/00135
USPC 600/103

(57) ABSTRACT

The insertion tube of an endoscope is equipped with an array of pressure sensors that provide real-time information of the pressure exerted by the insertion tube as it passes through a patient's body during an endoscopic procedure. Pressure information may be displayed in real time, wherein regions of high pressure are highlighted. An alarm is generated when the pressure applied at any location inside the patient's body exceeds safe limits. Further, sensors may be placed along the length of the insertion tube for measurement of distance travelled by the tip of an endoscope, which is integrated with measurement of the force applied by a physician in maneuvering the endoscope to determine applied force at a given location.

### CROSS-REFERENCE TO RELATED APPLICATIONS

The present specification relies on U.S. Provisional Patent Application No. 61/886,572, entitled “Endoscope with Integrated Location Determination”, and filed on Oct. 3, 2013, for priority.

In addition, the present specification relies on U.S. Provisional Patent Application No. 61/890,881, entitled “Endoscope with Integrated Pressure Sensing”, and filed on Oct. 15, 2013, for priority.

The present specification further relies on U.S. Provisional Patent Application No. 61/980,682, entitled “System and Method for Monitoring the Position of A Bending Section of An Endoscope”, and filed on Apr. 17, 2014, for priority.

All of the above-mentioned applications are herein incorporated by reference in their entirety.

### FIELD

The present specification relates generally to endoscopes, and more specifically, to methods and systems for sensing pressure and preventing perforations during endoscopic procedures.

### BACKGROUND

Endoscopes have attained great acceptance within the medical community, since they provide a means for performing procedures while at the same time enabling the physician to view the internal anatomy of the patient. Over the years, numerous endoscopes have been developed and categorized according to specific applications, such as cystoscopy, colonoscopy, laparoscopy, and upper GI endoscopy among others. Endoscopes may be inserted into the body's natural orifices or through an incision in the skin.

An endoscope typically comprises an elongated tubular shaft, rigid or flexible, having a video camera or a fiber optic lens assembly at its distal (or inspection) end. The shaft is connected to a handle, which sometimes includes an ocular for direct viewing. Visualization is also possible via an external screen. Various surgical tools may be inserted through a working channel in the endoscope for performing different surgical procedures.

When using an endoscope, a common problem is to be able to maneuver the inspection end of the scope and position it in proximity to the area of interest. This maneuvering is performed by a trained operator, who uses a combination of visual inspection of images and tactile coordination to maneuver through the twists and turns found in the GI system. The operator subjectively senses the resistance to maneuvers by the “feel” of the instrument and anticipates the amount of force necessary to advance the endoscope shaft forward. The application of force to the colon and its anatomic attachments can be painful. The frequent, albeit undesirable, occurrence of excessive contact pressure on an internal tissue can result in pain and in some cases, in perforation.

Colonoscopic injuries as a result of applying pressure by the scope lead to significant patient discomfort and increased treatment and litigation cost. The number of patients that undergo endoscopic screening is large therefore injury to colon or other internal tissues—even if infrequent—results in a large injured population. In particular, the task of inserting the insertion section of the endoscope into the large intestine is a complex one, because the large intestine itself has a complex shape and further, the shape of the large intestine varies from patient to patient. Thus, while inserting and maneuvering the endoscope through the large intestine, precision is required. Also, adjustments are required in the insertion distance (distance travelled by the endoscope through the lumen) and the amount of force used to achieve proper results in an endoscopic procedure.

Endoscopic injuries may result from a variety of the different portions of the scope used for a given procedure. For example, “looping”, which occurs when the rate at which the flexible shaft of the endoscope advances is greater than the speed at which the tip of the endoscope is advanced into the patient, is one of the main causes for perforation(s) during a procedure. When this occurs the flexible shaft can loop upon itself, which can exert pressure on the wall of a patient's lumen that is sufficient to cause a perforation.

As explained above, in endoscopic procedures, the physician has limited information on the pressure being placed inside the patient's body. U.S. Pat. No. 8,033,991, assigned to Artann Laboratories, Inc., describes “[a] handgrip for a colonoscope shaft comprising: an internal sleeve adapted to be placed over and engaged with said colonoscope shaft, an external sleeve slidingly positioned about the internal sleeve, and an engaging means positioned between said internal and external sleeves, said engaging means further equipped with a force sensor, a torque sensor and an acceleration sensor, said force sensor adapted to measure force applied along said colonoscope shaft, said torque sensor adapted to measure rotational torque applied to said colonoscope shaft, and said acceleration sensor adapted to measure acceleration of motion of said handgrip.”

U.S. Pat. No. 7,931,588, also assigned to Artann Laboratories, Inc. describes “[a] comprehensive system for objective assessment of colonoscope manipulation includes a handgrip for collecting and transmitting colonoscope handling data including force and motion data; a patient pain monitor for collecting and transmitting data on the level of patient's pain and discomfort; and digital processing means for extracting useful features such as colonoscope tip advancement speed from colonoscope-provided video images. All data is wirelessly transmitted to an electronic unit for processing and displaying on a monitor. A colonoscopy procedure is properly conducted when certain shaft advancement causes appropriate tip advancement, all without an increased level of patient's pain. The system of the invention is aimed at providing objective assessment data allowing for safer and less painful colonoscopies.”

The systems and devices described above, however, do not provide a method for measuring the actual pressure being exerted within the patient's body. Providing the physician with information on pressure hotspots will not only reduce injuries, but also reduce the fear of endoscopic or colonoscopic screening in patients.

Therefore, there is a need in the art for endoscopes that provide information to the physician about the pressure being exerted inside a patient's lumen at any given point and time during an endoscopic procedure. There is also a need for an endoscopic device and method that allows for immediate correction of applied pressure, in case it is too high, to prevent any injuries to the patient's lumen. There is further a need in the art for endoscopes that provide information to the physician about the distance travelled by the endoscope and the exact location of the distal tip within the patient's lumen. This would assist the physician with both quickly annotating a spot where an anomaly is found and determining the correct amount of pressure to apply depending on the location of the endoscope within the lumen.

### SUMMARY

The insertion tube of an endoscope is equipped with an array of pressure sensors that provide real-time information of the pressure exerted by the insertion tube as it passes through a patient's body during an endoscopic procedure. A GUI is provided for displaying the pressure information in real time, wherein regions of high pressure are highlighted. An alarm is generated when the pressure applied at any location inside the patient's body exceeds safe limits. In another embodiment, sensors are placed along the length of the insertion tube for measurement of distance travelled by the tip of an endoscope, which is integrated with measurement of force applied by the physician in maneuvering the endoscope to determine applied force at a given location. In one embodiment, the insertion tube comprises a series of vertebrae, each equipped with pressure sensors. The vertebrae can be individually moved to adjust pressure at a given point inside the patient's lumen.

In some embodiments, the present specification discloses an endoscope system capable of determining a distance travelled by a tip section of an endoscope from an entrance site into a patient's body through the patient's body, the endoscope system comprising: an insertion tube terminating in the tip section, said tip section comprising a plurality of viewing elements; a plurality of sensors located on an external surface of the insertion tube, wherein each of said plurality of sensors has a non-transitory memory and wherein each of said plurality of sensors has embedded, in its non-transitory memory, a unique identifier; and a depth sensor adapted to be positioned outside the patient's body, at the entrance site, wherein said depth sensor is configured to detect one of said plurality of sensors and wherein said detected sensor is a sensor that is positioned on the insertion tube closest to the entrance site relative to all other sensors of the plurality of sensors.

Optionally, the plurality of sensors are placed in predefined increments across substantially an entire length of the insertion tube. Still optionally, the plurality of sensors are embedded into the external surface of the insertion tube. Still optionally, the plurality of sensors are embedded into a removable, pliable sheet wherein said removable, pliable sheet is configured to be placed over the insertion tube.

Optionally, the unique identifier of each of said plurality of sensors is indicative of a sensor distance from the tip of the insertion tube.

In some embodiments, the endoscope system further comprises a processing unit adapted to receive said unique identifier from the depth sensor and, using said unique identifier, determine a distance travelled by the tip of the insertion tube within the patient's body.

In some embodiments, the endoscope system may further comprise a display, wherein said processing unit generates a distance image indicative of the determined distance travelled by the tip of the insertion tube within the patient's body and transmits said distance image to the display. Optionally, the display is configured to display said distance image alongside an endoscopy image.

In some embodiments, the plurality of sensors comprise at least one sensor for every five centimeters of the insertion tube.

Optionally, the plurality of sensors on the insertion tube may comprise any one of inductive sensors, capacitive sensors, capacitive displacement sensors, photoelectric sensors, magnetic sensors, or infrared sensors.

In some embodiments, the present specification discloses an endoscope system capable of determining a distance travelled by a tip section of an endoscope from an entrance site into a patient's body through the patient's body, the endoscope system comprising: an insertion tube terminating in the tip section, said tip section comprising a plurality of viewing elements; a plurality of markings positioned on an external surface of the insertion tube, wherein each of said plurality of markings represents a unique identifier; an imaging device adapted to be positioned outside the patient's body, at the entrance site, wherein said imaging device is configured to capture one of said plurality of markings and wherein said detected marking is a marking that is positioned on the insertion tube closest to the entrance site relative to all other markings of the plurality of markings; and, a processing unit adapted to receive an image of the detected marking from said imaging device and, using said image of the detected marking, determine a distance travelled by the tip of the insertion tube within the patient's body, wherein said processing unit generates a distance image indicative of the determined distance travelled by the tip of the insertion tube within the patient's body.

Optionally, the plurality of markings are placed in predefined increments across substantially an entire length of the insertion tube. In some embodiments, said plurality of markings may comprise at least one marking for every five centimeters of the insertion tube.

Optionally, the unique identifier of each of said plurality of sensors is indicative of a distance from the tip of the insertion tube.

In some embodiments, the endoscope system may further comprise a display, wherein said processing unit transmits said distance image to the display and wherein the display is configured to display said distance image alongside an endoscopy image.

In some embodiments, the endoscope system may further comprising a handle connected to the insertion tube, wherein the handle comprises an actuation device which, when activated, transmits a signal to the processing unit to store a distance measurement corresponding to an endoscopy image. In some embodiments, the present specification is directed toward an endoscope system configured to minimize a risk of perforating a patient's gastrointestinal tract during an endoscopic procedure, said endoscope comprising: a tip section, said tip section comprising a plurality of viewing elements to generate front and side views; an insertion tube connected to said tip section; a plurality of pressure sensors positioned on a surface of the insertion tube, wherein each of said pressure sensors is configured to generate data indicative of a pressure being experienced at a surface of the insertion tube corresponding to said each pressure sensor; a processing unit configured to receive said pressure data, compare said pressure data to one or more threshold pressure data levels, and generate an alarm if said pressure data exceeds a predetermined amount.

Optionally, said plurality of pressure sensors are distributed over substantially an entire length of the insertion tube. Still optionally, at least one pressure sensor is positioned at least every five centimeters over said surface of the insertion tube. Still optionally, the plurality of pressure sensors is placed around a circumference of the insertion tube. Still optionally, the plurality of pressure sensors are embedded into an external surface of the insertion tube. And still optionally, the plurality of pressure sensors are embedded into a removable, pliable sheet and wherein said removable, pliable sheet is configured to be placed over the insertion tube.

In some embodiments, the endoscope system further comprises a display, wherein said processing unit is configured to generate a pressure image that comprises a color-coded representation of an endoscope movement through the patient's body and wherein different colors correspond to different levels of pressure.

In some embodiments, the present specification is directed toward an endoscope system configured to minimize a risk of perforating a patient's gastrointestinal tract during an endoscopic procedure, said endoscope comprising: a tip section, said tip section comprising a plurality of viewing elements to generate front and side views; an insertion tube connected to said tip section, wherein the insertion tube comprises a series of cylindrical sections, each section capable of moving in three dimensions; a plurality of pressure sensors positioned in the insertion tube and associated with at least one of said cylindrical sections, wherein each of said pressure sensors is configured to generate data indicative of a pressure being experienced at the associated at least one cylindrical section; a processing unit configured to receive said pressure data, compare said pressure data to one or more threshold pressure data levels, and generate an alarm if said pressure data exceeds a predetermined amount; and a controller that, in response to said pressure data, causes one or more cylindrical sections to move when said pressure data exceeds said predetermined amount.

Optionally, a movement of each cylindrical section is individually controllable by the controller. Still optionally, the controller is configured to cause a cylindrical section to move in a direction which results in a decrease of pressure. Still optionally, the movement is angular. Still optionally, the movement is translational.

Optionally, if said pressure data exceeds the predetermined amount, the controller is configured to cause a cylindrical section associated with a pressure level exceeding said predetermined amount to move in a direction which results in a decrease of pressure.

In some embodiments, the present specification is directed toward an endoscope system configured to minimize a risk of perforating a patient's gastrointestinal tract during an endoscopic procedure, said endoscope comprising: a tip section, said tip section comprising a plurality of viewing elements; an insertion tube connected to said tip section, wherein the insertion tube comprises a series of cylindrical sections, each section capable of moving in three dimensions; a plurality of pressure sensors positioned in the insertion tube and associated with at least one of said cylindrical sections, wherein each of said pressure sensors is configured to generate data indicative of a pressure being experienced at the associated at least one cylindrical section; a handheld device in data communication with at least one of said plurality of pressure sensors, wherein said handheld device is configured to receive pressure data from the at least one of said plurality of pressure sensors; and a processing unit configured to receive said pressure data, compare said pressure data to one or more threshold pressure data levels, and generate an alarm if said pressure data exceeds a predetermined amount.

Optionally, the handheld device is a glove comprising at least one sensor to determine an applied force.

Optionally, said plurality of pressure sensors are distributed over substantially an entire length of the insertion tube. Still optionally, at least one pressure sensor is positioned at least every five centimeters over said surface of the insertion tube.

In some embodiments, each of the plurality of pressure sensors may have a unique identifier embedded therein and wherein said unique identifier is indicative of a distance of said each of the plurality of pressure sensors from a distal end of the insertion tube.

In some embodiments, the endoscope system may further comprise a depth sensor adapted to be positioned outside the patient's body, at an entrance site, wherein said depth sensor is configured to detect a distance of one of said plurality of pressure sensors and wherein said detected pressure sensor is a pressure sensor that is positioned on the insertion tube closest to the entrance site relative to all other sensors of the plurality of pressure sensors. Optionally, said system further comprises a display, wherein said processing unit is configured to generate a pressure image that comprises a color-coded representation of an endoscope movement through the patient's body and wherein different colors correspond to different levels of pressure.

The aforementioned and other embodiments of the present shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 illustrates an endoscopy system;

FIG. 2 is an illustration of three major zones of the colon where applied force or pressure is measured;

FIG. 3 depicts an embodiment of an endoscope insertion portion that includes integrated pressure sensors;

FIG. 4a illustrates an external sleeve with integrated sensors that can be used with an endoscopic insertion tube, according to one embodiment;

FIG. 4b illustrates the external sleeve with integrated sensors of FIG. 4a when placed on an endoscopic insertion tube;

FIG. 5 illustrates a bending diameter of insertion tube, according to one embodiment of the present specification;

FIG. 6 illustrates one embodiment of an endoscope with integrated sensors;

FIG. 7 is an exemplary GUI (Graphical User Interface) display, showing a graph of pressure hotspots caused by an insertion portion in a color-coded fashion;

FIG. 8 is another exemplary GUI display, showing a bar graph of pressure hotspots caused by an insertion portion throughout different areas of the colon, in a color-coded fashion;

FIG. 9 is another exemplary GUI display, showing a graph of applied pressure or force data caused by an insertion portion throughout different areas of the colon, as presented to a physician during a procedure;

FIG. 10a is a block diagram illustrating overall system architecture, according to one embodiment of the present specification;

FIG. 10b is a block diagram illustrating overall system architecture, according to one embodiment of the present specification;

FIG. 11 illustrates a depth sensor placed at the entrance of a patient's lumen or body cavity;

FIG. 12 illustrates a depth measurement using the depth sensor shown in FIG. 11, according to one embodiment;

FIG. 13 is a block diagram illustrating a depth sensing system, according to one embodiment;

FIG. 14 illustrates a process of marking or recording a measured depth value, according to one embodiment;

FIG. 15 is an illustration of tick marks or numbers on an insertion tube, representing markers for identifying the location of the scope within the body;

FIG. 16 illustrates an imaging device placed at the entrance of the body;

FIG. 17 illustrates depth measurement using the imaging device shown in FIG. 16, according to an embodiment;

FIG. 18 shows an exemplary set of data illustrating the range of force applied at the three major zones of colon during colonoscopy procedures;

FIG. 19 illustrates a glove with force sensors integrated therein, according to one embodiment of the present specification;

FIG. 20 is a block diagram illustrating a system for measuring applied force relative to a given point within the body, according to one embodiment of the present specification;

FIG. 21 illustrates common sites of perforation during a colonoscopy procedure;

FIG. 22 illustrates an insertion tube comprised of vertebra portions, according to one embodiment;

FIG. 23 is a block diagram illustrating a system for adjusting the vertebra portions, shown in FIG. 22, in response to feedback from corresponding pressure sensors, according to one embodiment of the present specification;

FIG. 24A illustrates a perspective view of a bending section of a multi-viewing element endoscope;

FIG. 24B illustrates another view of the bending section shown in FIG. 24A; and

FIG. 24C illustrates a perspective view of a tubular segment of the bending section shown in FIG. 24A.

### DETAILED DESCRIPTION

In one embodiment, the present specification discloses an endoscope with pressure sensors integrated along the part of the scope which is inserted into the patient's body cavity. The sensors provide real-time information on the pressure being applied at various parts of the patient's lumen, and this information is available on the display associated with the endoscope. This type of real-time feedback allows the physician to naturally and dynamically adjust the pressure they are applying. In one embodiment, pressure is automatically adjusted to prevent perforation or injury to the patient's lumen. In one embodiment, an audible alert and/or a visual alert, such as flashing lights on the display screen are activated when high pressure is detected, to draw the attention of the physician.

In another embodiment, the present specification is directed towards methods and systems for determining the distance travelled by the tip of an endoscope inside a patient's body. In one embodiment, the insertion tube of an endoscope is equipped with sensors, each sensor having a unique identifier, code, signature, or other identification according to its distance from the tip of the insertion tube. A depth sensor placed outside the patient's body at the entrance site of the insertion tube detects the sensor on the tube closest to the entrance site to provide an indication of the distance travelled by the tip of the endoscope. In another embodiment, an imaging device is placed at the entrance site of the insertion tube, and it captures the image of the marking on the insertion tube closest to the entrance site. In another embodiment, measurement of distance travelled by the tip of an endoscope is integrated with measurement of force applied by the physician in maneuvering the endoscope. An alarm is generated when the force applied relative to a location inside the patient's body exceeds safe limits.

In one embodiment, real-time information is provided by the integrated sensors on the distance being travelled by the endoscope inside the patient's lumen, and this information is available on the display associated with the endoscope. This kind of real-time feedback allows the physician to naturally and dynamically determine the location of the endoscope tip, mark any spots with anomalies and adjust the pressure they are applying.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

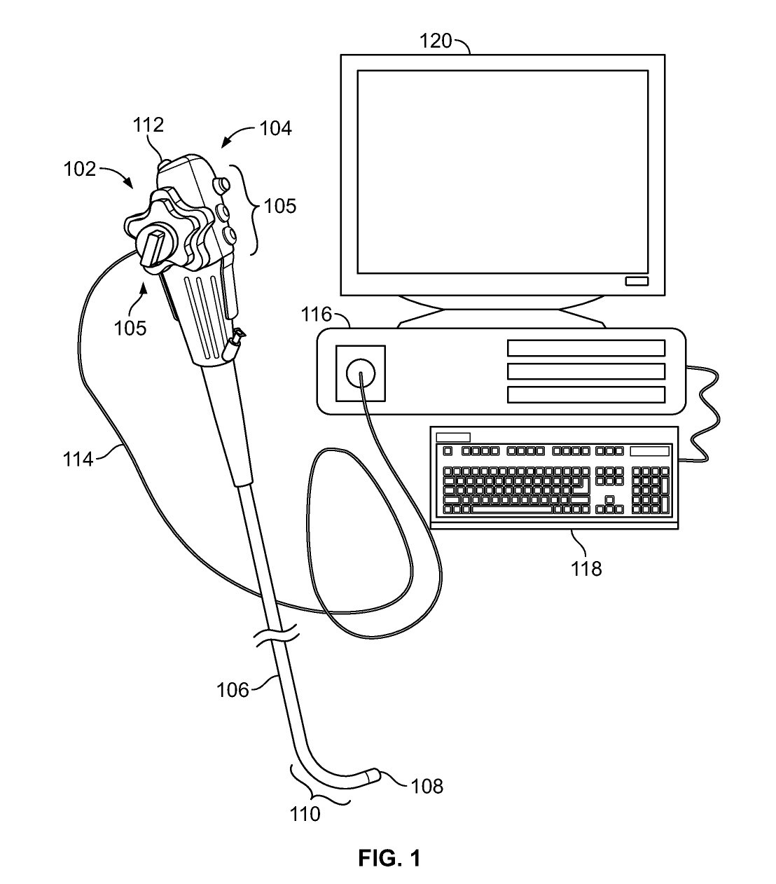

Reference is now made to FIG. 1, which shows an endoscopy system 100. System 100 includes a single or multi-viewing element endoscope 102. Endoscope 102 further includes a handle 104, from which an elongated shaft or insertion tube 106 emerges. Elongated shaft 106 terminates with a tip section 108 which is turnable by way of a bending section 110. The tip section 108 comprises a front-pointing viewing element for generating a front view, and in a multi-viewing element endoscope it further comprises one or more side-pointing viewing elements for generating side views, as the elongated shaft or insertion tube moves through a patient's body. Handle 104 is used for maneuvering elongated shaft 106 within a body cavity; the handle also includes one or more knobs and/or switches 105 which control bending section 110 as well as functions such as fluid injection and suction. Handle 104 further includes one or more service channel opening 112 through which surgical tools may be inserted.

A utility cable 114 connects between handle 104 and a controller 116. Utility cable 114 includes therein one or more fluid channels and one or more electrical channels. The electrical channel(s) include at least one data cable for receiving video signals from the front and side-viewing elements, as well as at least one power cable for providing electrical power to the viewing elements and to the discrete illuminators.

Controller 116 governs power transmission to the endoscope's 102 tip section 108, such as for the tip section's viewing elements and illuminators. Controller 116 further controls one or more fluid, liquid and/or suction pump which supply corresponding functionalities to endoscope 102. One or more input devices, such as a keyboard 118, are connected to controller 116 for the purpose of human interaction with the controller. In another configuration (not shown), an input device, such as a keyboard, may be integrated with the controller in a same casing.

A display 120 is connected to controller 116, and configured to display images and/or video streams received from the viewing element (s) of the endoscope 102. Display 120 may further be operative to display a user interface for allowing a human operator to set various features of system 100.

Optionally, the video streams received from the different viewing elements of multi-viewing element endoscope 102 may be displayed separately on display 120, either side-by-side or interchangeably (namely, the operator may switch between views from the different viewing elements manually). In another configuration (not shown), two or more displays are connected to controller 116, each for displaying a video stream from a different viewing element of the multi-viewing element a endoscope.

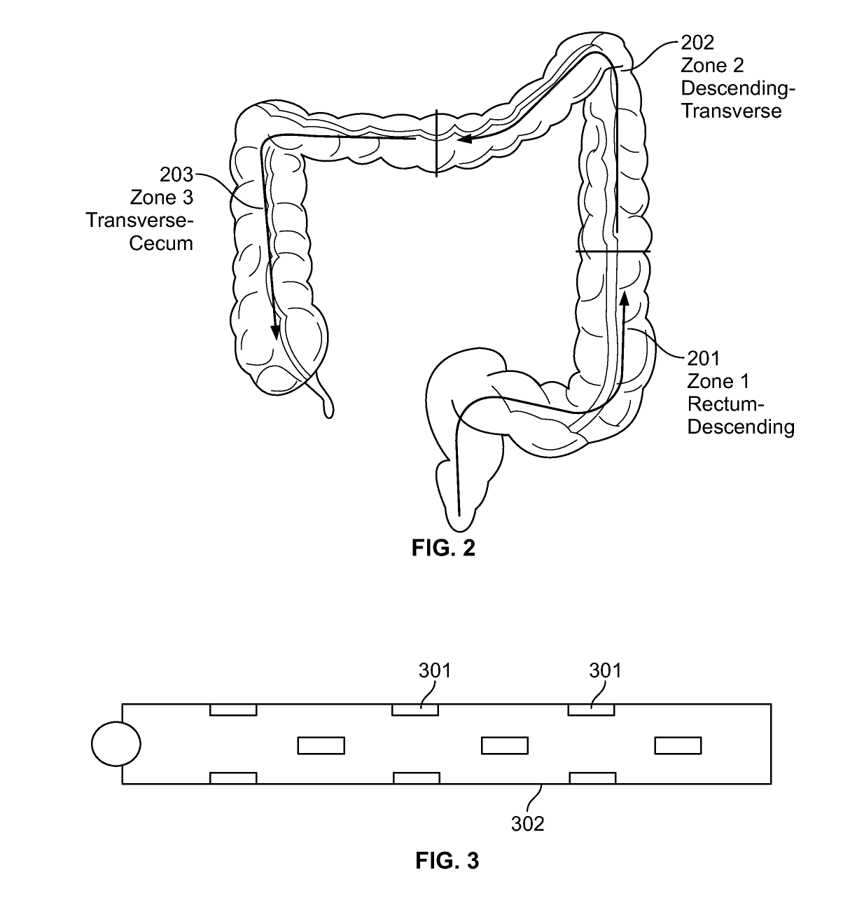

During an endoscopic procedure, such as colonoscopy, force or pressure is applied when the endoscope is pushed into the colon, specifically while entering the colon, and also while withdrawing the endoscope from the colon (pulling). FIG. 2 illustrates three major zones of the colon to focus upon when measuring the forces of pushing or pulling. Referring to FIG. 2, the three zones are rectum-descending 201, which has a length range of around 60.5 cm, descending-transverse 202 having a length range of around 30 cm, and transverse-cecum 203 with a length range of around 47.5 cm. It should be noted that the average colon is approximately 1.5 m long—the anal canal is approximately 5 cm; the rectum is approximately 12 cm; the sigmoid colon is approximately 40 cm; the descending colon is approximately 15 cm; the transverse colon is approximately 45 cm; and the ascending colon is approximately 25 cm. These length ranges also provide an approximation of the distance travelled by the insertion tube of an endoscope inside the body.

One of ordinary skill in the art would appreciate that only a limited amount of pressure can be applied to portions of a patient's GI tract before it may rupture. For example, a normal human intestine may be ruptured by the application of 210.5 mmHg of pressure or more. A patient's sigmoid colon may be ruptured by the application of 169 mmHg of pressure or more.

In one embodiment, and as illustrated in FIG. 3, the present invention employs an array of pressure sensors placed throughout the scope's body, to measure the actual pressure exerted inside the patient's lumen during an endoscopic procedure. Pressure sensors 301 are placed along the elongated shaft or insertion tube 302 of the endoscope, shown earlier as component 106 in FIG. 1. In one embodiment, pressure sensors are adapted to be used with all of types of scopes.

In one embodiment, in order to not limit or interfere with the natural flexibility of the insertion tube, the pressure sensors are installed on the outer circumference of the insertion tube. Further, the placement of pressure sensors has minimal effect on the outer diameter of the insertion tube. This is enabled in one embodiment, by using a pressure gauge matrix which is incorporated into the outer layer of the insertion tube, thereby allowing sensitive readings without effecting tube flexibility or requiring a wider tube diameter. In one embodiment, the pressure gauge matrix provides a net of sensors along the insertion tube, wherein each sensor is synchronized with the other sensors to provide a real time map of the pressure put inside the lumen. This real time synchronization between all sensors in a matrix provides an advantage over using individual sensors.

In one embodiment, pressure sensors may be placed externally on the insertion tube by using mechanical means, such as screws. In another embodiment, sensors may be mechanically connected, welded, glued, molded, adhered, or otherwise attached on the external side of the tube.

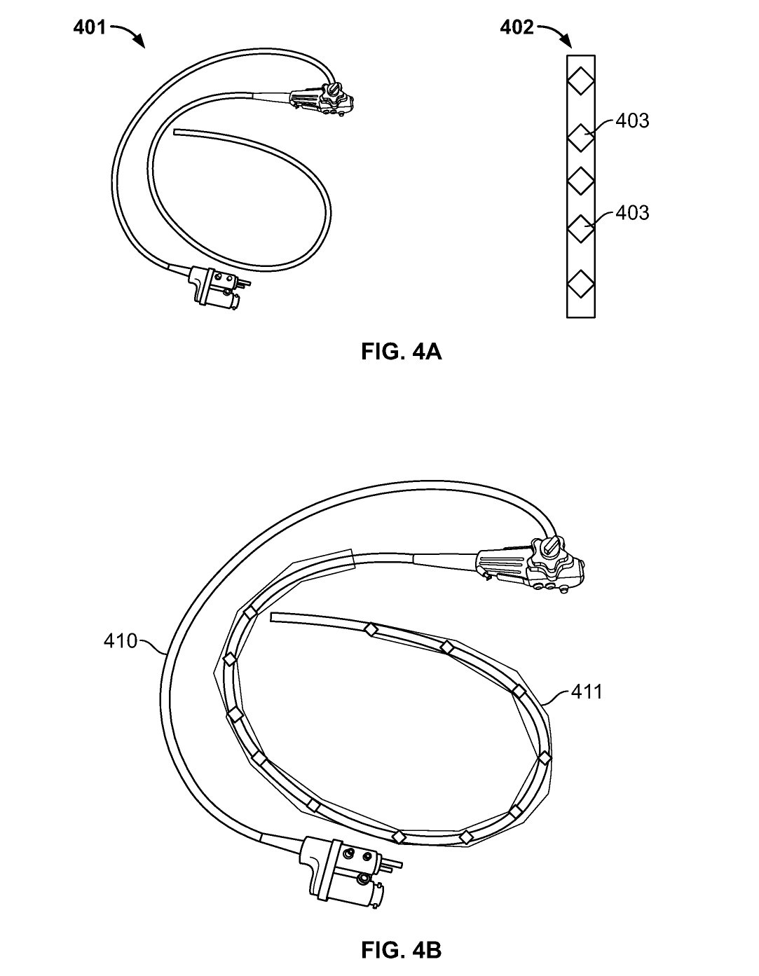

In another embodiment, pressure sensors are integrated into an external sleeve slipped over the insertion tube before an endoscopic procedure, as shown in FIGS. 4a and 4b. Referring to FIG. 4a, an endoscope without sensors 401 is shown next to a sleeve 402, which has sensors 403 integrated into it. Referring to FIG. 4b, endoscope 410 is shown with the sleeve with sensors 411 put on. One of ordinary skill in the art would appreciate that the sleeve with sensors 411 is placed in such a manner such that it covers substantially the entire length of the insertion tube that is passed through the patient's body. The sleeve 411 can be removed from the tube 410 at the end of the procedure. In one embodiment, the sleeve is disposable. The disposable sleeve, which was slipped over the insertion tube before an endoscopic procedure, can be removed from the tube at the end of the procedure. Further, the sleeve with sensors can be used with any type of endoscope.

In another embodiment, a pressure gauge matrix is incorporated into the outer layer/jacket of the tube, in a similar fashion to the external sleeve described above.

One of ordinary skill in the art would appreciate that pressure sensors are employed on the outer side of the scope, and not inside in order to measure the pressure applied by the insertion tube at specific locations of the body during procedure.

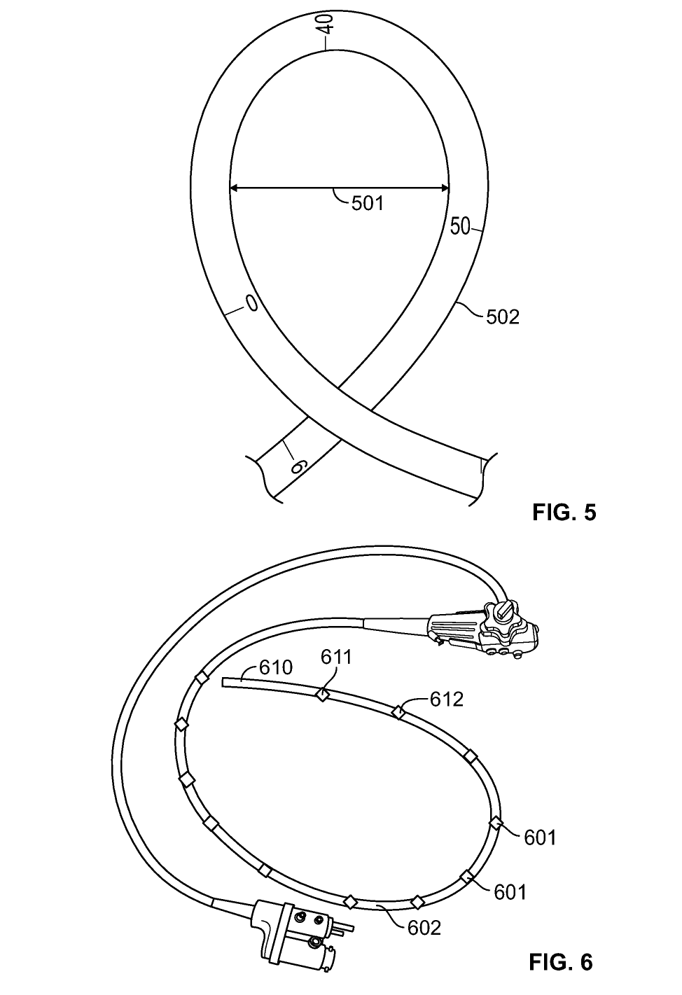

In one embodiment, the pressure sensors are placed in a manner such that it covers substantially the entire length of the insertion tube that is passed through the patient's body. It may be noted that the bending diameter of the insertion tube is typically of the order of 10-20 centimeters. Bending diameter of the insertion tube is defined as the diameter of the loop which is formed when the insertion tube is bent. FIG. 5 illustrates the bending diameter 501 of the insertion tube 502.

Keeping in mind the bending diameter therefore, in one embodiment, a pressure sensor is placed periodically and repeatedly a predefined distance, for example, every 1, 2, 3, 4, 5, 6, 7, 8, 9, 10, 11, 12, 13, 14, 15, 16, 17, 18, 19, or 20 centimeters, or any increment therein, along the length of the insertion tube. This embodiment is illustrated in FIG. 6. Referring to FIG. 6, sensors 601 are placed along the elongated shaft or insertion tube 602 of the endoscope, also shown earlier as component 106 in FIG. 1. in this example, and not limited to such example, a sensor 611 would be placed at a distance of 10 centimeters from the distal end 210 of the tube, the next sensor 612 at a distance of 20 centimeters, and so on. For a tube length of around 150 centimeters, the number of sensors employed would be 150/10=15. Further, in order to enable circumferential monitoring, the total number of sensors required would be 15*3=45. This ensures that the entire periphery of tube is covered.

In one embodiment, the number of sensors is determined per unit length of the insertion tube. In one embodiment, a minimum of one sensor per every five centimeters of tube length is employed. In one embodiment, a maximum of one sensor per every one centimeter of tube length is employed.

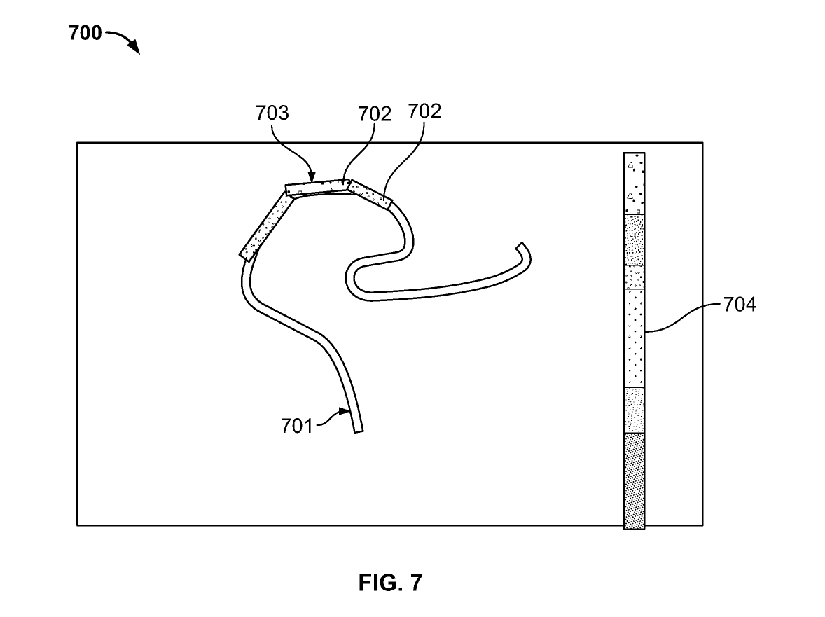

The pressure sensors employed on the endoscope provide real time information about the pressure being applied by the insertion tube at various locations inside the patient's lumen, as the scope advances. In one embodiment, the present invention provides a graphical user interface (GUI) on the display of the endoscope system, which presents data from the pressure sensors in an easily understandable manner to the physician (endoscopist). FIG. 7 illustrates an exemplary GUI display 700, wherein a curve 701 simulating the endoscope's movement through the patient's GI tract. In one embodiment, various regions 702 of the curve are color-coded to represent corresponding pressure exerted by the endoscope at that region. Thus, for example, low pressure may be indicated by blue color, while a high pressure hot-spot 703 may be indicated by red. In one embodiment, a color bar 704 is provided on the GUI display, to provide a reference to the physician to determine the various ranges of pressure corresponding to different colors.

In one embodiment, on the basis of pressure measurements provided by the sensors on the endoscope, if the applied pressure exceeds the typical maximum values in a procedure, it is not only indicated visually on the GUI display, but also by generating an audible alarm or beep, prompting the physician to immediately correct the pressure they are applying. In one embodiment, visual indication of high pressure includes flashing in red color the relevant region in the curve that represents the movement of endoscope tube.

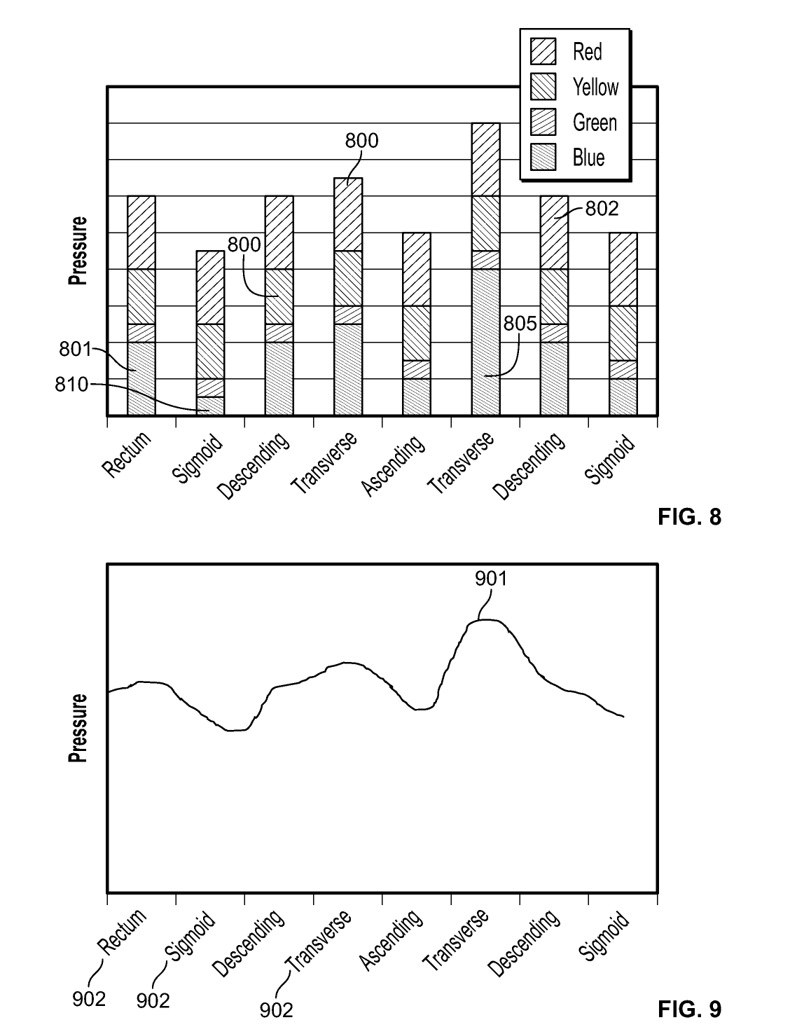

FIG. 8 illustrates another GUI screen, wherein the pressure applied is presented as a function of location during a colonoscopy procedure, since a physician applies very different pressures to move through different parts of the colon, such as rectum, sigmoid, ascending, descending and transverse regions. Referring to FIG. 8, regions 801 in the histogram bars 800 represent the pressure typically applied by the physician during procedure with respect to the location in the colon. Regions 802, on the other hand, indicate high pressure being applied in a certain location in the colon, and is be used to generate an alarm. In one embodiment, such data is recorded and used for statistical analysis and also for the purpose of training the endoscope operators. For example, it can be seen from FIG. 8, that the highest value of “normal” pressure (regions 801) is permissible in the transverse region 805, whereas the sigmoid region 810 is considered sensitive to pressure and herein the normal pressure has the minimum value. In one embodiment, the various regions are color coded to indicate different pressure values. For example, in one embodiment, typically applied pressure regions 801 are represented in blue. In another embodiment, high pressure regions 802 are represented by red, green, and/or yellow to represent different high pressures that may cause an alarm to sound.

In one embodiment, stored pressure data from endoscopic procedures is periodically analyzed to determine typical limits of normal pressure and the range of pressure values to generate an alarm, for various regions of the lumen.

In one embodiment, pressure data is stored during each procedure and logged with the name of the specific physician conducting the endoscopic procedure. Whenever the same physician conducts the next procedure, the system retrieves their previous record for reference and guidance. The system also computes the average values of pressure for the procedures conducted by a specific physician and uses that to generate an alert if the pressure being exerted during a procedure exceeds the physician's average.

FIG. 9 illustrates another exemplary GUI screen, by which the applied pressure may be presented to the physician performing the endoscopy. The pressure curve 901 corresponds to pressures at various regions 902 of the colon, such as sigmoid, rectum, transverse, etc. as explained earlier. In one embodiment, an estimate of relative distance between the distal tip and the entry point of the insertion tube, co-related with the display image, is used to determine various regions of the colon.

It may be noted that in case of a multi camera endoscope with corresponding number of displays, pressure data in the form of color bar, graph or color-coded representation of endoscope movement through the body, may be displayed in either one or all of the displays, as chosen by the physician. One of ordinary skill in the art would also appreciate that apart from the stated examples, measured pressure data may be presented to the physician conducting the procedure in any manner that is easy to comprehend and draws immediate attention in case the pressure applied is above normal.

Pressure measurements provided by the sensors on the endoscope are received by the main control unit or controller of the system (shown as 116 in FIG. 1). Data regarding tolerable or maximum limits of pressure is also uploaded to the main control unit. Thus the control unit provides the user with a graphic display illustratively showing pressure exerted by the insertion tube inside the lumen, and also generates an alarm when the pressure exceeds the maximum limit.

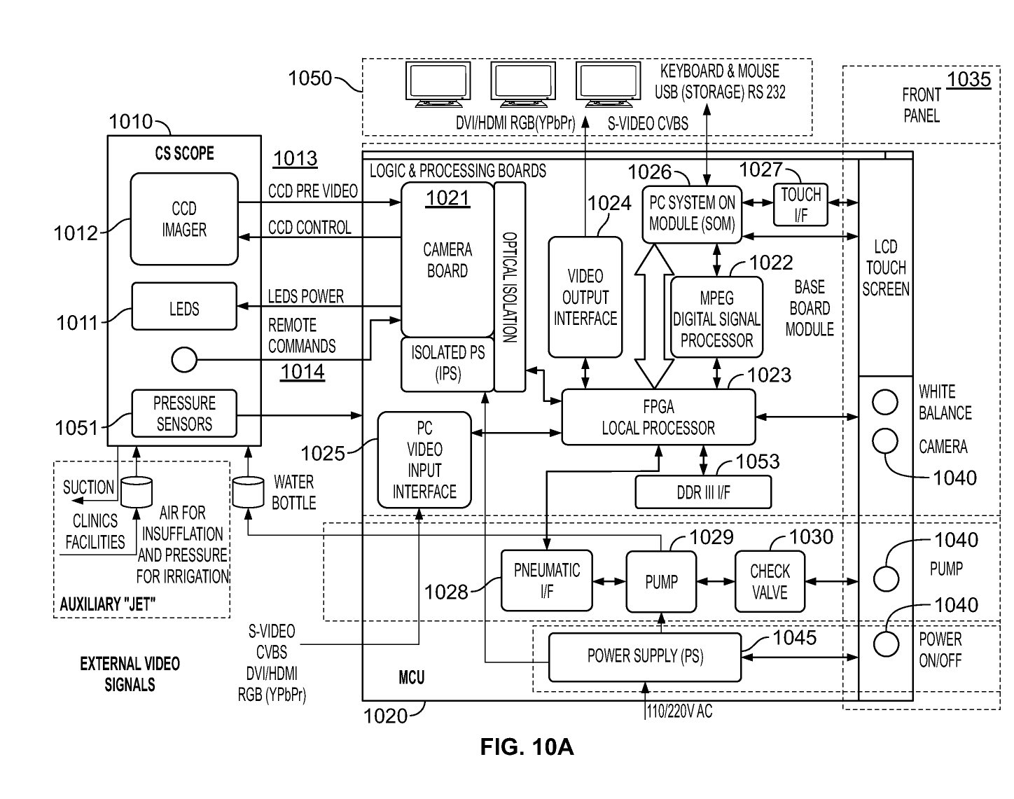

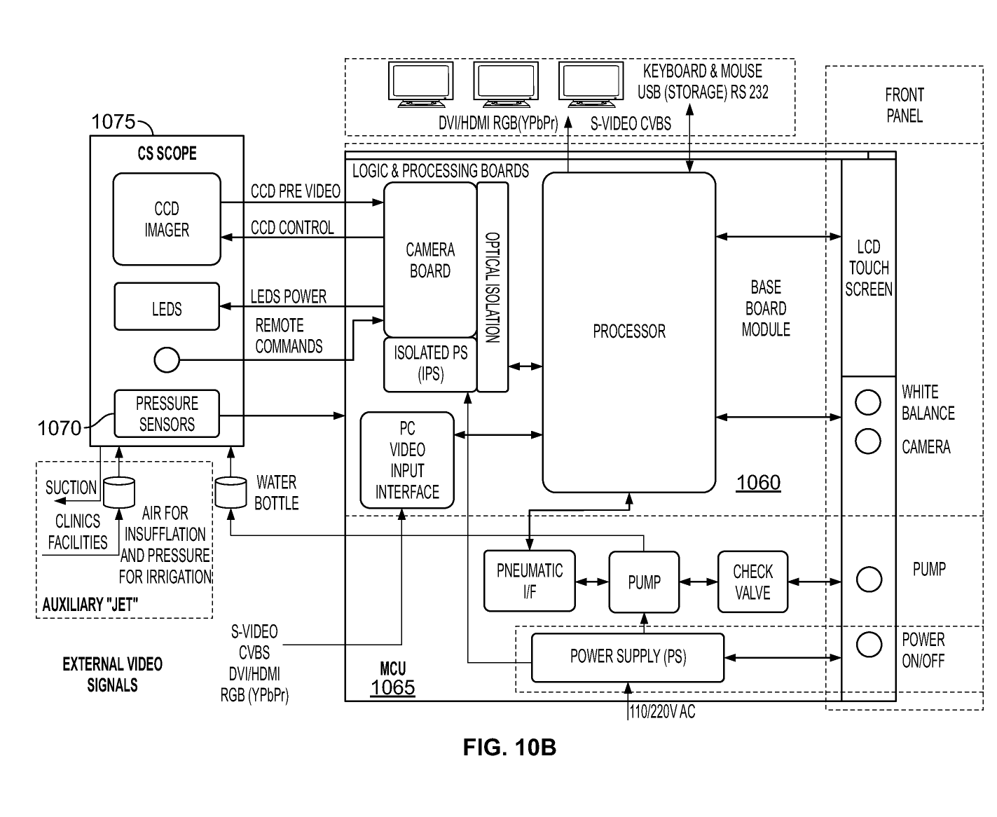

FIG. 10a details how the main control unit (MCU) or the controller 1020 operatively connects with the endoscope 1010 and the display units 1050. Referring to FIG. 10a, endoscope 1010 comprises pressure sensors 1051, image sensors 1012 and associated LEDs 1011. Data from the pressure sensors is supplied to the MCU 1020, where it is processed for display on the GUI.

Controller circuit board 1020 further comprises a camera board 1021 that transmits appropriate commands to control the power supply to the LEDs 1011 and to control the operation of image sensor 1012 (comprising one or more cameras) in the endoscope. The image sensor or camera may be a Charge Coupled Device (CCD) or a Complementary Metal Oxide Semiconductor (CMOS) imager. The camera board in turn receives video signal 1013 generated by the CCD imager and also other remote commands 1014 from the endoscope.

Controller circuit board 1020 further comprises elements for processing the video obtained from the imager 1012, including MPEG Digital Signal Processor 1022 and FPGA local processor 1023. The FPGA 1023 is responsible for video interpolation and on-screen display overlay prior to sending the video to the MPEG DSP 1022. The FPGA acts as a main controller of the system for image processing, video writing and on-screen display, and generates the various GUI screens for presenting real time pressure information. In one embodiment, pressure data from the sensors 1051 in the endoscope is stored in the DDR memory 1053 associated with the FPGA 1023. This data is used by the FPGA to compute average maximum values and generate alarms if the average maximum values of pressure are exceeded.

After processing by the FPGA, the video signal is sent for display through video output interface 1024. A video input interface 1025 is also provided for receiving video input from an external video source. In one embodiment, the video input comprises analog video, such as in CVBS, S-Video or YPBPR format or digital video (DVI), and is displayed as such.

The system on module (SOM) 1026 provides an interface to input devices such as keyboard and mouse, while the touch I/F 1027 provides touch screen interface. The controller 1020 further controls one or more fluid, liquid and/or suction pump(s) which supply corresponding functionalities to the endoscope through pneumatic I/F 1028, pump 1029 and check valve 1030. The controller further comprises a power supply on board 1045 and a front panel 1035 which provides operational buttons 1040 for the user.

In another embodiment, the pressure sensors 1051 transmit measurement data directly to the FPGA 1023 which processes the information. Alarm signals indicating high pressure are sent to the GUI through the SOM 1026. The FPGA 1023 can send information to a video output interface 1024 and then to the displays 1050.

In another embodiment, the pressure sensors 1051 transmit measurement data directly to the SOM 1026, which processes the information and generates alarm signals if required. The alarm signals are then sent to the GUI and/or to a remote screen/monitor.

In another embodiment shown in FIG. 10b, a single processor 1060 located on the MCU 1065 is used to process the information from pressure sensors 1070 located on the scope 1075. In one embodiment, the FPGA and SOM are not involved in this process.

Besides alerting a physician in case the applied pressure exceeds safe limits, in one embodiment, the present invention also includes a mechanism of automatically lowering the applied pressure when it exceeds the tolerable limits.

In another embodiment, the present specification discloses an endoscope with sensors integrated along insertion tube that provide real-time information on the distance being travelled by the endoscope inside the patient's lumen. This information is available on the display associated with the endoscope. This kind of real-time feedback allows the physician to naturally and dynamically determine the location of the endoscope tip, mark any spots with anomalies and adjust the pressure they are applying.

In one embodiment, the present invention employs an array of sensors placed throughout the scope's body, to determine the depth that the insertion tube travels inside the patient's lumen during an endoscopic procedure. In one embodiment, as shown and described earlier with reference to FIG. 6, sensors 601 are placed along the elongated shaft or insertion tube 602 of the endoscope, also shown earlier as component 106 in FIG. 1. Further, each sensor has a unique identifier, code, signature, or other identification according to its location (such as distance from the distal tip) along the elongated axes of the insertion tube. Thus for example, and not limited to such example, a sensor would be placed at a distance of 1, 2, 3, 4, 5, 6, 7, 8, 9, 10, 11, 12, 13, 14, 15, 16, 17, 18, 19, or 20 centimeters, or any increment therein, from the distal end of the tube. The next sensor may be placed at a similar, or different, distance and would have an identifier that is different than the identifier programmed into the first sensor. In another embodiment, each identifier is not only unique to the sensor but also indicative of the particular position, or distance, occupied by the sensor. Thus, in one embodiment, a plurality of sensors are placed at 10 centimeter increments along the length of the insertion tube, where each sensor has a different identifier and where each identifier is indicative of the distance increment occupied by the sensor. In one embodiment, the number of sensors is determined per unit length of the insertion tube. In one embodiment, a minimum of one sensor per every five centimeters of tube length is employed. In one embodiment, a maximum of one sensor per every one centimeter of tube length is employed. Further, the sensors are adapted to be used with all of kinds of endoscopes. Several different types of sensors may be employed, including, but not limited to inductive sensors, capacitive sensors, capacitive displacement sensors, photoelectric sensors, magnetic sensors, and infrared sensors.

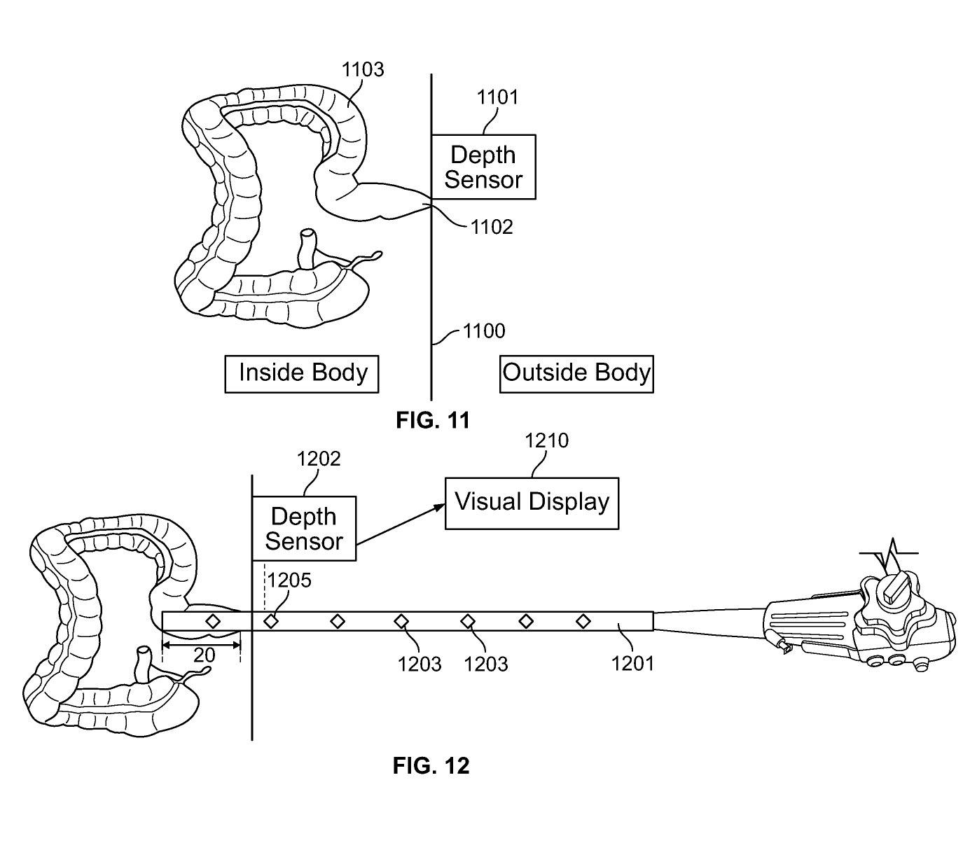

Additionally, a depth sensor is placed at the entrance of the body where the endoscope is inserted and is in communication with the display that is used with the endoscope. This is illustrated in FIG. 11. Referring to FIG. 11, the depth sensor 1101 is placed outside the body 1100, close to the rectum 1102, which is the entry point for an endoscope into the colon 1103.

The operation of depth sensor with respect to the sensors located on the endoscope is shown by the way of example in FIG. 12. Referring to FIG. 12, in this example, the insertion tube 1201 of the endoscope is about 20 cm inside the body. Sensors 1203 are placed at regular distances on the insertion tube, as also described earlier with reference to FIG. 6. In present example, each sensor is placed 10 cm apart. In operation, the depth sensor 1202 detects alignment to sensor 1205 closest to the entrance site, outside the body. In this case, the closest sensor 1205 is at a 20 centimeters from the tip of the endoscope, and this is indicated by the depth sensor. In one embodiment, each sensor is pre-programmed to be read according to its location, such that the 10 cm sensor would transmit a different output than the 20 cm sensor. In one embodiment, the output of the depth sensor is conveyed to the controller of the system, which provides an appropriate visual indication on the display 1210 of the distance travelled by the distal end of the scope.

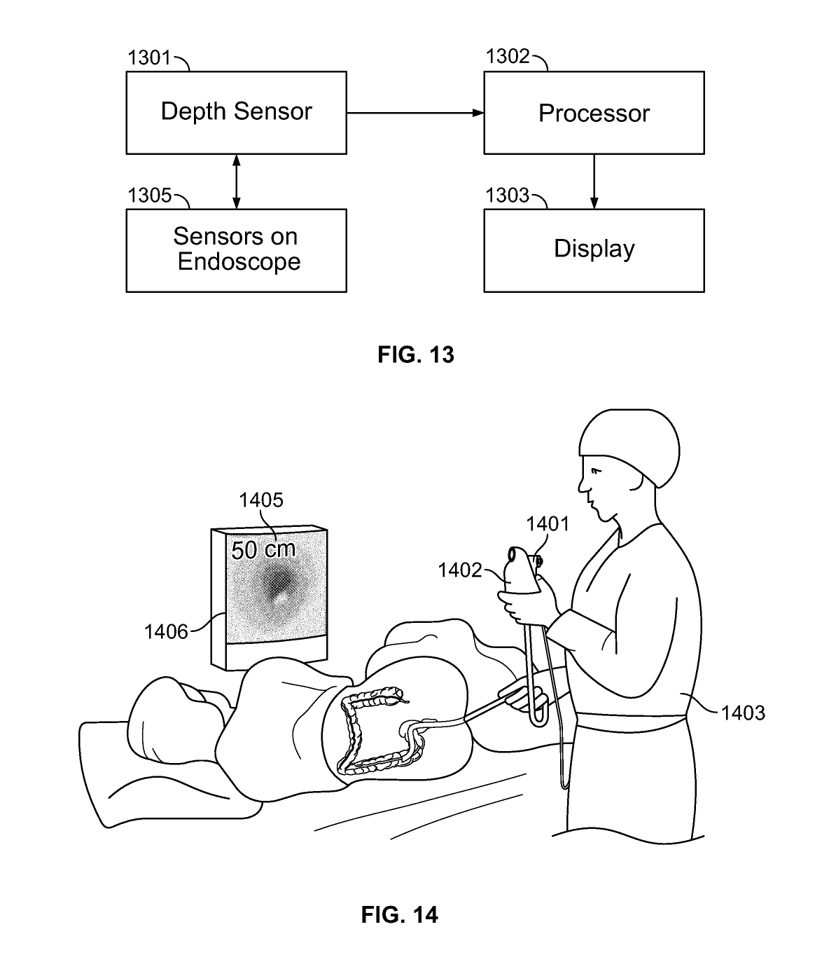

In another embodiment shown in FIG. 13, the depth sensor 1301 provides output to a separate processor 1302, which is connected to a display 1303. The depth sensor detects the alignment with the appropriate sensors 13013 on the endoscope, and corresponding information regarding the distance travelled by the endoscope inside the patient's body is provided on the display.

In one embodiment, the present invention employs a matrix of sensors, so that continuity in reading of distances is achieved.

In one embodiment, sensors are placed inside the insertion tube. Further, sensors are attached into the internal wall of the insertion tube by any suitable mechanical means, such as by using screws or any other mechanical connector. One the advantages of placing sensors internally in the insertion tube, is that it provides resistance to reprocessing and allows scope washing without affecting the sensors. Further, attaching sensors by mechanical means provides for ease of manufacturing.

In another embodiment, in order to not limit or interfere with the natural flexibility of the insertion tube, sensors are placed on the external side of the insertion tube. Further, the placement of sensors has minimal effect on the outer diameter of the insertion tube. Further in this embodiment, touch sensors may be used. Thus, for example, with touch sensors placed at regular intervals on the insertion tube, the number of touch sensors showing an output would indicate the depth the insertion tube has travelled inside the lumen.

In one embodiment, sensors may be placed externally on the insertion tube by using mechanical means, such as screws. In another embodiment, sensors may be glued on the external side of the tube. In another embodiment, sensors are integrated into an external sleeve threaded on the insertion tube before an endoscopic procedure, such as that as shown and described with reference to FIGS. 4a and 4b for pressure sensors.

The present invention provides the real time information on the display with regards to the distance travelled by the endoscope. This allows the physician conducting the endoscope to focus on the procedure and saves them the hassle of computing the distance inside the body if an anomaly is found. In one embodiment, the handle comprises an actuation device which, when activated, transmits a signal to the processing unit to store a distance measurement corresponding to an endoscopy image. In some embodiments, the actuation device may be a button, switch, touchpad, or any other input device.

In one embodiment, as shown in FIG. 14, an actuation device in the form of a button 1401 is provided on the endoscope handle 1402, which can be pressed by the physician 1403 to “mark” a spot of interest during the procedure. Activating the button sends a signal to the controller of the endoscope system (shown in FIG. 1), to record the current distance 1405 as measured and displayed on the screen 1406. Thus, the physician does not have to remember the distance for a point of interest, and is assisted by the system when required to re-examine the tissue or perform a procedure. In one embodiment, when the tip of the endoscope is returned to the marked position, the display notifies the user of the proximity to the marked location.

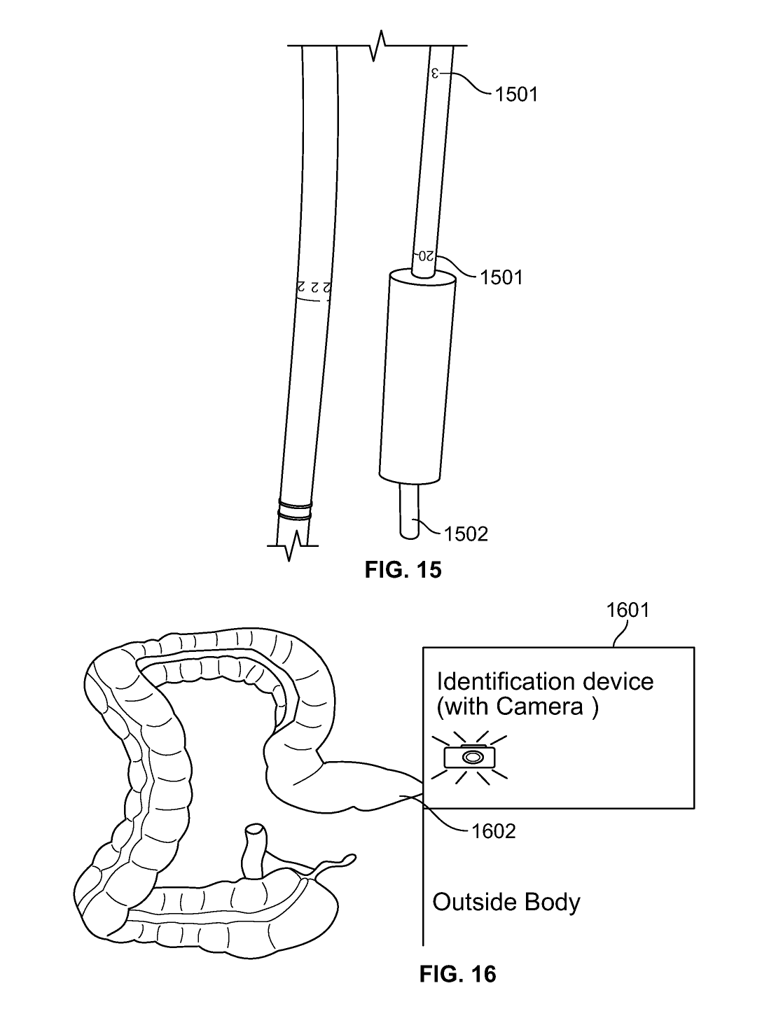

It is known in the art that the insertion tube has numbers on it to indicate to the physician the distance of the insertion tube within patient body. This is shown in FIG. 15, wherein 1501 are number markings on the insertion tube, indicating the location of the scope in the body, with respect to the tip of the insertion tube 1502. The aim of the present invention, however, is to eliminate the need for the physician to calculate and remember and/or record the distance inside the body for the pathologic findings relative to the insertion tube location.

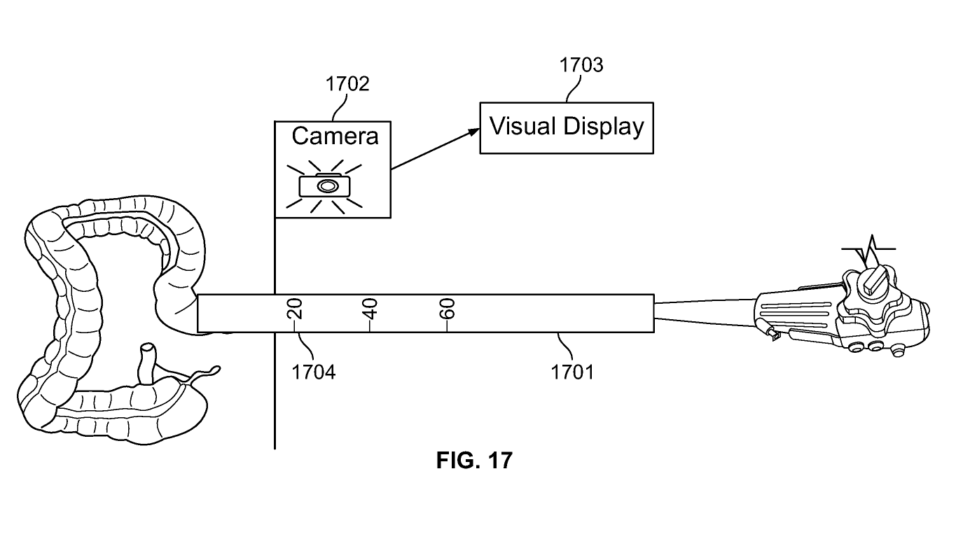

Thus, in another embodiment, the present invention uses a camera or another imaging device, such as a CCD to read the number markings on the insertion tube. This is illustrated in FIG. 16. Referring to FIG. 16, the imaging device 1601 is placed outside the patient's body, close to the entrance point 1602 of the insertion tube of the endoscope. The operation of imaging device is shown by the way of an example in FIG. 17. Referring to FIG. 17, in this example, the insertion tube 1701 of the endoscope is about 20 cm inside the body. The imaging device 1702 captures the “20 cm” mark 1704 on the endoscope, and displays the result on an associated display 1703. In one embodiment, the imaging device is completely external to the endoscopic system, and is associated with its own processor/controller and display. In one embodiment, the controller of the imaging device is programmed to have the imaging device continuously capture images and send the output to the display. In one embodiment, the display unit is common to the imaging device and the endoscope system. In another embodiment, the imaging device is completely integrated with the endoscope system. This may be implemented, for example, by connecting the imaging device to the endoscope handle. Further, buttons may be provided on the endoscope handle to control the operation of the imaging device. The output of the imaging device is provided to the endoscope controller, along with the output of the image sensors located in the endoscope tip. In one embodiment, the output of the imaging device (number marking) is displayed alongside the image generated by the endoscope. In one embodiment, the image device may be controlled from the endoscope handle, the keyboard (shown as 118 in FIG. 1), or by a touch screen display associated with the controller. In one embodiment, the image device is controlled by a separate touch screen and processor.

In one embodiment, depth is measured by using sensors that respond to the physician's grip on the tube. Sensors are placed over substantially the entire length of the insertion tube, and each sensor has a unique identifier, code, signature, or other identification per its location along elongated axes of the insertion tube. Thus for example, if the physician is holding the tube around the “40 cm” mark, the corresponding sensor at that point responds to the physician's hold, to indicate that the tube is being held at 40 cm. Further, since the typical distance between the point that the physician holds the tube and the body cavity is about 20 cm, this distance can be subtracted from the hold location to obtain an estimate of the depth of the insertion tube inside the body. Thus, in the present example, depth would be 40−20=20 cm, approximately. In one embodiment, an activation device is employed such that the sensors respond only to user's (physician's) hold and activation of sensors on the insertion tube in response to pressure or touch inside the lumen is avoided.

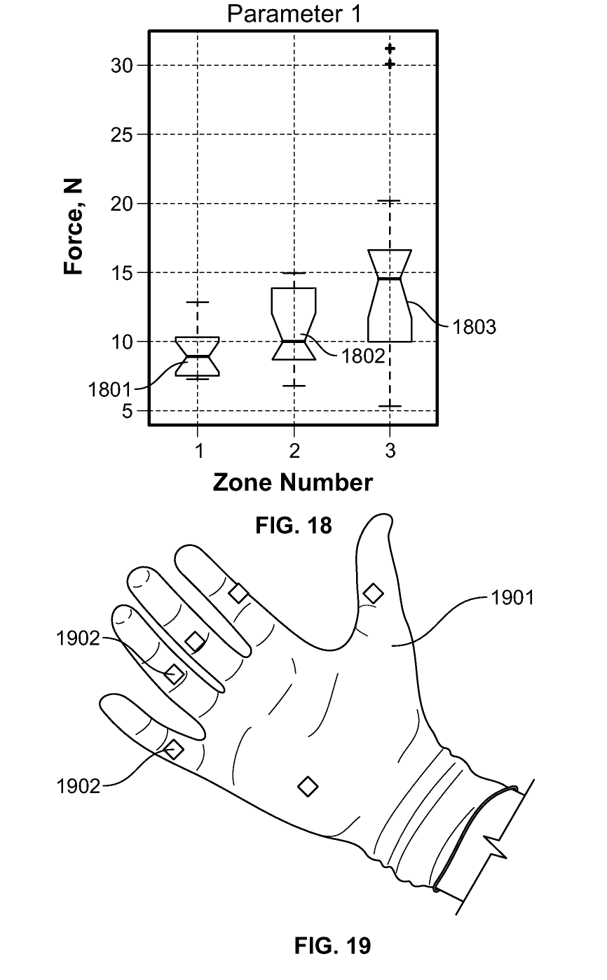

In one embodiment, the present invention allows a user to determine, and in response, control the force or pressure applied by the insertion tube relative to specific locations inside the body during an endoscopic procedure. As described earlier with reference to FIG. 2, during an endoscopic procedure, such as colonoscopy, force or pressure is applied when the endoscope is pushed into the colon, that is, while entering the colon, and also while withdrawing from the colon (pulling). The three major colon zones are rectum-descending, descending-transverse, and transverse-cecum. FIG. 18 illustrates an exemplary set of data illustrating the range of force applied at the three major zones of colon during colonoscopy procedures. Referring to FIG. 18 in conjunction with FIG. 2, in Zone 1 (rectum-descending) 1801, the maximum applied force ranges from 10-13 N in a typical colonoscopy. For Zone 2 (descending-transverse) 1802, the maximum force is typically in the range of 14-15 N; and for Zone 3 (transverse-cecum) 1803, the maximum force is typically in the range of 23-32 N. In one embodiment, 2.7-9 kg of force is required for colon perforation.

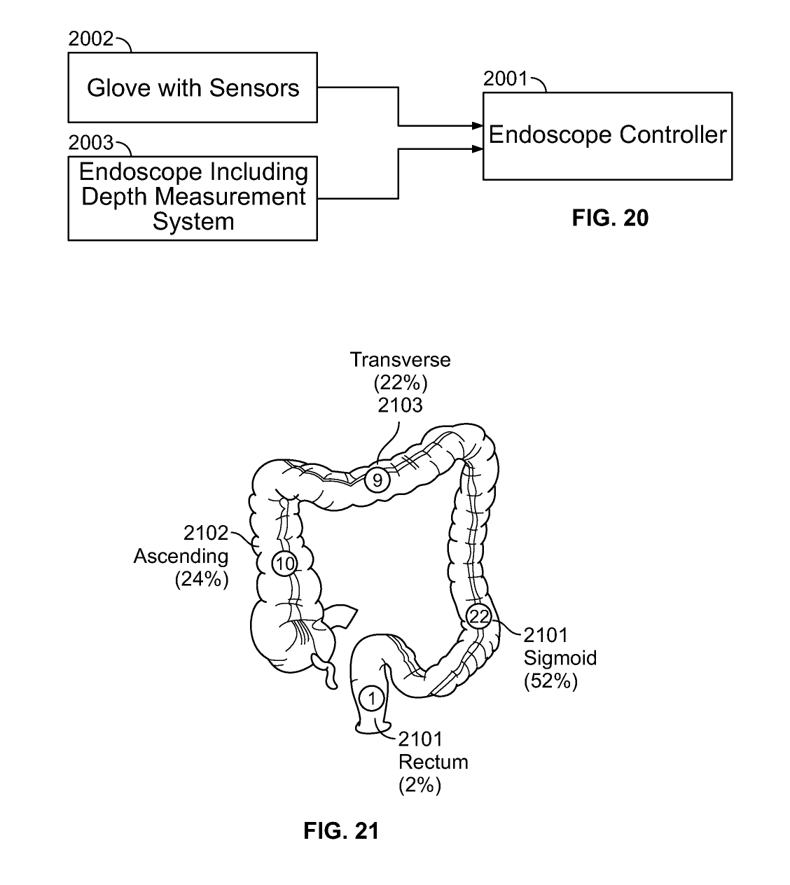

In order to ensure that the force applied by a physician conducting the endoscopic procedure does not exceed the typical maximum for a given region, in one embodiment the present invention uses a glove integrated with a sensor system to measure the amount of force used to push or pull the endoscope. This glove is shown in FIG. 19. Referring to FIG. 19, glove 1901 can be worn by the physician at the time of performing an endoscopic procedure. In one embodiment, the glove is disposable. Sensors 1902 on the glove measure the applied force and send the output to a processor or controller by wired or wireless means. In one embodiment, sensors 1902 transmit measured force data to the controller of the endoscope. This embodiment is illustrated in FIG. 20. Endoscope controller 2001 receives data from the sensors on the glove 2002. Further, the controller 2001 also receives data from the endoscope 2003 and its depth-measuring system in accordance with embodiments described in FIGS. 12 and 17. The controller co-relates the two sets of data—current applied force and current depth of the endoscope, to determine if the force being applied is in appropriate range for a given location inside the body

In one embodiment, on the basis of force measurements provided by the sensors on the glove, if the applied force exceeds the typical maximum values for a given location in a procedure, it is indicated visually on the display associated with the endoscope system, such as by a warning flash. In one embodiment, an audible alarm or beep is also generated, prompting the physician to immediately correct the force they are applying.

In one embodiment, the controller provides the user with a graphic display illustratively showing force exerted by physician relative to the position of the insertion tube inside the lumen. The graphical display may be in the form of a color graph as shown in FIG. 7, or in the form of a curve as shown in FIG. 9. In one embodiment, the Graphical User Interface (GUI) screen displays a histogram, such as that shown in FIG. 8 to illustrate the force typically applied by the physician during procedure with respect to the location in the colon, as well as the amount of force being applied in the current procedure. In one embodiment, the force typically applied or the normal range of force applied are highlighted in a different color compared to the force presently being applied. In one embodiment, such data is recorded and used for statistical analysis and also for the purpose of training the endoscope operators. One of ordinary skill in the art would appreciate that apart from the stated examples, applied force and depth data may be presented to the physician conducting the procedure in any manner that is easy to comprehend and draws immediate attention in case the force applied is above normal.

In one embodiment, applied force data is stored during each procedure and logged with the name of the specific physician conducting the endoscopic procedure. Whenever the same physician conducts the next procedure, the system retrieves their previous record for reference and guidance. The system also computes the average values of force for the procedures conducted by a specific physician and uses that to generate an alert if the force being applied during a procedure exceeds the physician's average.

It is known in the art that the most common site for perforation amongst various segments of the colon is Sigmoid zone of colon, as illustrated in FIG. 21. Referring to the figure, the highest number (about 52% of the total) perforations take place in S-shaped sigmoid region 2101, followed by the ascending region 2102, transverse region 2103 and rectum 2104. The reasons for this include sharp angulation in the sigmoid region and its freedom of movement which makes it susceptible to displacement. Common diverticular formation of the sigmoid region and pelvic adhesions in the patient's lumen due to prior inflammation or operations are other factors to be considered for the risk of perforation when performing an endoscopic procedure.

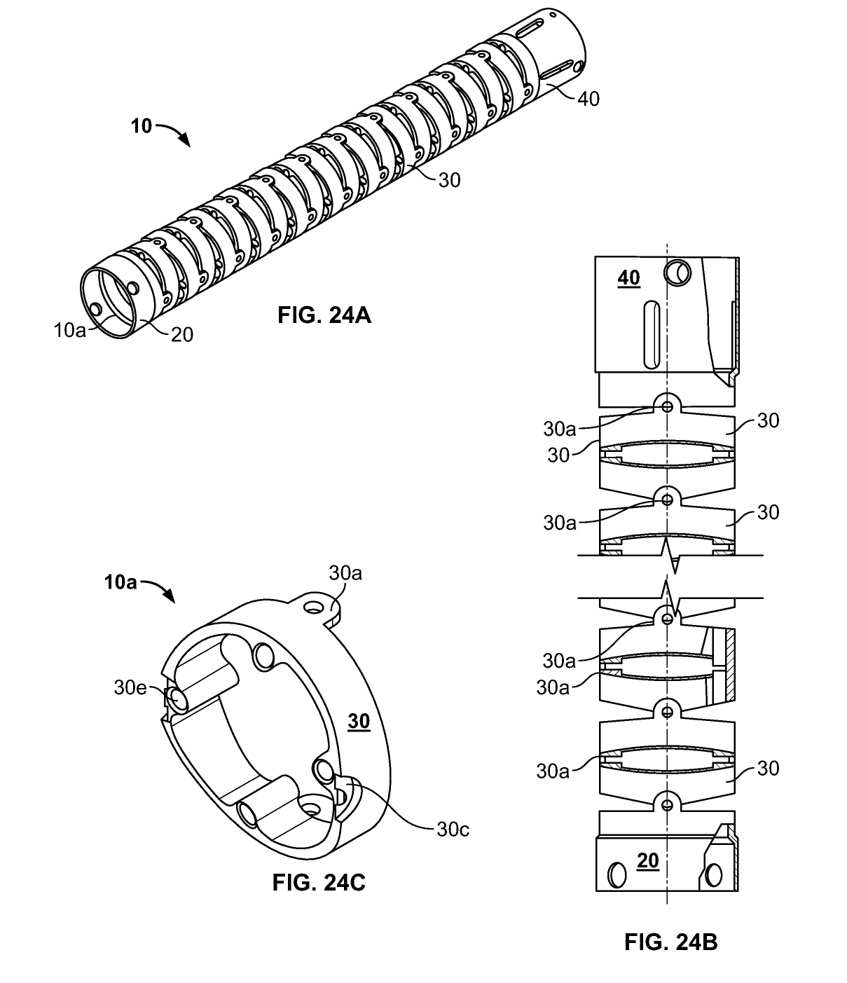

FIG. 24A illustrates a perspective view of a bending section of a multi-viewing element endoscope. The bending section 10 comprises a plurality of mutually articulated tubular segments 20, 30, 40. In some embodiments, the proximal articulated segment 20 and the distal articulated segment 40 are constructed differently than the segment 30. A lumen 10a runs through the bending section 10.

FIG. 24B illustrates another view of the bending section shown in FIG. 24A. As is shown, segment 30 comprises at least one tubular part. The at least one tubular part comprises two oppositely oriented axially extending tabs 30a positioned along a first periphery and one or more recesses (not shown) positioned along a second opposing periphery. The segments 30 are coupled together by tabs 30a of a first tubular part which fits into a recess of an adjacent second tubular part. For example, as shown in FIG. 24B, tab 30a of first part 30 fits into a recess (not shown) of second part 30, and so forth, thereby coupling segment parts 30.

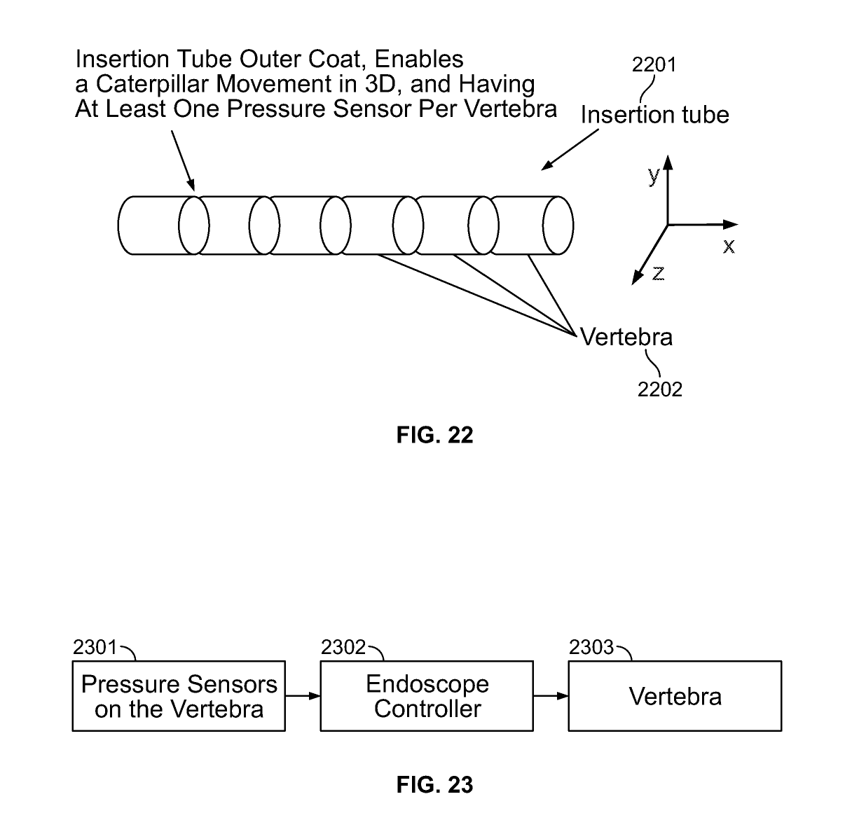

FIG. 24C illustrates a perspective view of the segment 30 of the bending section, shown in FIG. 24B. In an embodiment, segment 30 comprises one or more axially extending tabs 30a positioned along a distal periphery. In an embodiment, segment 30 comprises one or more recesses 30c positioned along a proximal periphery for coupling with extending tabs 30a of segment 30. Thus, one or more axially extending tabs 30a are provided on a first segment 30 for coupling with a recess 30c of a second, adjacent segment 30. Cable guides 30e are positioned along internal walls of segment 30, extending into lumen 10a (shown in FIG. 24A). In various embodiments one or more steering cables may be threaded through these cable guides to enable the maneuvering of bending section 10. In order to provide ease of mobility of the insertion tube during a procedure and to reduce the risk of perforations, in one embodiment the insertion tube (shown as 106 in FIG. 1) is designed such that it comprises a series of several cylindrical sections. As is known in the art, the insertion tube is a hollow tube through which all the electronic cables/wires, working channel/s, water and air channels pass. In the present embodiment as shown in FIG. 22, the insertion tube 2201 comprises a series of cylindrical sections 2202, known as vertebrae. Each vertebra 2202 can move in three dimensions along the x axis, y axis and the z axis. Thus, the vertebrae enable a simple yet effective movement of a particular section of the insertion tube in case any adjustment is required, without having to move the entire tube, which may be uncomfortable or painful for the patient. In that sense, the vertebrae enable the relevant section to become a “secondary bending section” and provide enhanced maneuverability. In one embodiment, the simplest movement of the insertion tube inside the body resembles a snake movement, such that the vertebra at the front moves first, followed by the successive vertebra at the back. In one embodiment, the comprehensive movement of the vertebrae in the insertion tube resembles a marionette movement, wherein each manipulation applied on one of the marionette strings—a vertebra in this case, causes changes in the location of all the marionette components.

It may be noted that on one end the vertebrae of the insertion tube are connected to the bending section and on the other end, to the scope handle. Further, all the vertebrae are connected to each other, such that when one vertebra moves, it causes its adjoining vertebrae to move as well.

In one embodiment, the movement of each vertebra can be controlled by the controller of the endoscope system. In one embodiment, the number of vertebrae varies from 4 to 180.

In one embodiment, at least one pressure sensor is placed on each vertebra. In one embodiment, the number of pressure sensors is determined depending on the outer diameter of the scope. Thus, for example and not limited to this example, 10 pressure sensors are placed in a given circumference of the tube per vertebra. As another example, 10 sensors may be placed in a given circumference every 10 centimeters along the length of the insertion tube. It may be appreciated that the number of sensors for given circumference may vary from one to ten, depending on the length of the insertion tube, number of vertebrae along the insertion tube, insertion tube outer diameter and the like.

In one embodiment, when the insertion tube is pressed against the colon, the pressure sensors on the corresponding vertebrae sense the pressure and transmit the measurement to the controller or main control unit of the endoscope. This is shown in FIG. 23. Referring to FIG. 23, from the measurement received from pressure sensors 2301, the endoscope controller 2302 determines if the pressure applied at any vertebra is too high and may cause a perforation. This causes the controller to generate an audio and/or visual alert, as explained earlier in the specification. In one embodiment, apart from generating an alert, the controller commands the specific vertebra 2303 on which the alerted sensor was activated to move automatically to reduce the pressure. This enables an automatic correction of pressure in real time during an endoscopic procedure. In one embodiment, the vertebra auto-correction movement is pre-programmed into the controller. In one embodiment, the vertebra auto-correction movement comprises an angular movement to adjust the angle of the insertion tube at that point. In one embodiment, the vertebra auto-correction movement comprises a translational movement to move the insertion tube forward or backward from a given point. It may be noted that both kinds of movements can be combined to achieve an angular and a longitudinal correction movement, resulting in a pull or push motion relative to the colon. In one embodiment, the position of more than one vertebra is changed by the controller to reduce pressure. As mentioned above, the process of adjustment of vertebrae occurs in real-time with the feedback system comprising the “read and respond” operation of the controller continuing during the entire procedure.

One of ordinary skill in the art would appreciate that the system for auto-correction movement of the vertebrae aids the physician in inserting and moving the insertion tube through a patient's colon. The auto-correcting vertebra and the corresponding pressure sensors lead the insertion tube into the colon, without requiring the physician to apply undue force on the insertion tube as it is pushed into colon walls.

In one embodiment, the auto-correction movement of the scope can be cancelled, if the physician does not want to use it during the procedure. In one embodiment, an on/off option by means of a button or a switch is provided on the scope handle or the main control unit. In another embodiment, an on/off option is provided on the touch-screen associated with the endoscope display.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An endoscope system configured to minimize a risk of perforating a patient's gastrointestinal tract during an endoscopic procedure, said endoscope comprising
a tip section, said tip section comprising a plurality of viewing elements to generate front and side views;
an insertion tube connected to said tip section;
a plurality of pressure sensors positioned on a surface of the insertion tube, wherein each of said pressure sensors is configured to generate data indicative of a pressure being experienced at a surface of the insertion tube corresponding to said each pressure sensor; and,
a processing unit configured to receive said pressure data, compare said pressure data to one or more threshold pressure data levels, and generate an alarm if said pressure data exceeds a predetermined amount.

2. The endoscope system of claim 1, wherein said plurality of pressure sensors are distributed over substantially an entire length of the insertion tube.

3. The endoscope system of claim 2, wherein at least one pressure sensor is positioned at least every five centimeters over said surface of the insertion tube.

4. The endoscope system of claim 3, wherein the plurality of pressure sensors is placed around a circumference of the insertion tube.

5. The endoscope system of claim 1, wherein the plurality of pressure sensors are embedded into an external surface of the insertion tube.

6. The endoscope system of claim 1, wherein the plurality of pressure sensors are embedded into a removable, pliable sheet and wherein said removable, pliable sheet is configured to be placed over the insertion tube.

7. The endoscope system of claim 1, further comprising a display, wherein said processing unit is configured to generate a pressure image that comprises a color-coded representation of an endoscope movement through the patient's body and wherein different colors correspond to different levels of pressure.

8. An endoscope system configured to minimize a risk of perforating a patient's gastrointestinal tract during an endoscopic procedure, said endoscope comprising
a tip section, said tip section comprising a plurality of viewing elements to generate front and side views;
an insertion tube connected to said tip section, wherein the insertion tube comprises a series of cylindrical sections, each section capable of moving in three dimensions;
a plurality of pressure sensors positioned in the insertion tube and associated with at least one of said cylindrical sections, wherein each of said pressure sensors is configured to generate data indicative of a pressure being experienced at the associated at least one cylindrical section;
a processing unit configured to receive said pressure data, compare said pressure data to one or more threshold pressure data levels, and generate an alarm if said pressure data exceeds a predetermined amount; and
a controller that, in response to said pressure data, causes one or more cylindrical sections to move when said pressure data exceeds said predetermined amount.

9. The endoscope system of claim 8, wherein a movement of each cylindrical section is individually controllable by the controller.

10. The endoscope system of claim 8, wherein the controller is configured to cause a cylindrical section to move in a direction which results in a decrease of pressure.

11. The endoscope system of claim 10, wherein the movement is angular.

12. The endoscope system of claim 10, wherein the movement is translational.

13. The endoscope system of claim 8, wherein, if said pressure data exceeds the predetermined amount, the controller is configured to cause a cylindrical section associated with a pressure level exceeding said predetermined amount to move in a direction which results in a decrease of pressure.

14. An endoscope system configured to minimize a risk of perforating a patient's gastrointestinal tract during an endoscopic procedure, said endoscope comprising
a tip section, said tip section comprising a plurality of viewing elements;
an insertion tube connected to said tip section, wherein the insertion tube comprises a series of cylindrical sections, each section capable of moving in three dimensions;
a plurality of pressure sensors positioned in the insertion tube and associated with at least one of said cylindrical sections, wherein each of said pressure sensors is configured to generate data indicative of a pressure being experienced at the associated at least one cylindrical section;
a handheld device in data communication with at least one of said plurality of pressure sensors, wherein said handheld device is configured to receive pressure data from the at least one of said plurality of pressure sensors; and
a processing unit configured to receive said pressure data, compare said pressure data to one or more threshold pressure data levels, and generate an alarm if said pressure data exceeds a predetermined amount.

15. The endoscope system of claim 14 wherein the handheld device is a glove comprising at least one sensor to determine an applied force.

16. The endoscope system of claim 14, wherein said plurality of pressure sensors are distributed over substantially an entire length of the insertion tube.

17. The endoscope system of claim 16, wherein at least one pressure sensor is positioned at least every five centimeters over said surface of the insertion tube.

18. The endoscope system of claim 14, wherein each of the plurality of pressure sensors has a unique identifier embedded therein and wherein said unique identifier is indicative of a distance of said each of the plurality of pressure sensors from a distal end of the insertion tube.

19. The endoscope system of claim 14, further comprising a depth sensor adapted to be positioned outside the patient's body, at an entrance site, wherein said depth sensor is configured to detect a distance of one of said plurality of pressure sensors and wherein said detected pressure sensor is a pressure sensor that is positioned on the insertion tube closest to the entrance site relative to all other sensors of the plurality of pressure sensors.

20. The endoscope system of claim 14, further comprising: a display, wherein said processing unit is configured to generate a pressure image that comprises a color-coded representation of an endoscope movement through the patient's body and wherein different colors correspond to different levels of pressure.
